(19) United States
(12) Patent Application Publication
(10) Pub. No.: US 2020/0405728 A1
(43) Pub. Date: Dec. 31, 2020

(54) METHODS OF PREVENTING AND TREATING HYPOGLYCEMIA IN TYPE 1 AND TYPE 2 DIABETES PATIENTS

(71) Applicant: Ferox Therapeutics LLC, Montclair, NJ (US)

(72) Inventor: Joyce B. HARP, Montclair, NJ (US)

(73) Assignee: Ferox Therapeutics LLC, Montclair, NJ (US)

(21) Appl. No.: 16/977,246

(22) PCT Filed: Feb. 28, 2019
(86) PCT No.: PCT/US2019/019970

§ 371 Date: Sep. 1, 2020

Related U.S. Application Data
(60) Provisional application No. 62/637,082, filed on Mar. 1, 2018.
(60) Provisional application No. 62/671,519, filed on May 15, 2018.

(51) Int. Cl. A61K 31/55; A61K 31/4545; A61P 3/10

(52) U.S. Cl. CPC A61K 31/55; A61K 45/06; A61P 3/10; A61K 31/4545

(57) ABSTRACT

Disclosed are methods for increasing glucagon secretion in response to exogenous insulin-induced hypoglycemia in patients with type 1 or type 2 diabetes. Also disclosed are methods of increasing glucagon secretion, and thus preventing or treating hypoglycemia, in patients with insulin producing tumors. The methods include administering a therapeutic amount of a histamine 1 receptor antagonist, a histamine 3 receptor antagonist, and/or a combination histamine 1/3 receptor antagonist. It may also be further advantageous to administer a therapeutic amount of a serotonin receptor antagonist in the methods disclosed herein.

### CROSS-REFERENCE TO PRIOR FILED APPLICATIONS

This application claims the benefit of United States Provisional Application Nos. 62/637,082 filed Mar. 1, 2018, and 62/671,519 filed May 15, 2018, both of which are expressly incorporated by reference herein in their entirety.

### FIELD OF THE INVENTION

The invention pertains to methods of treating or preventing hypoglycemia in mammals through the administration of antagonists of the histamine 1 receptor, histamine 3 receptor, or both the histamine 1 and histamine 3 receptors.

### BACKGROUND OF THE INVENTION

In healthy individuals, blood glucose concentration is maintained within a very narrow range, despite major fluctuations in glucose intake and glucose utilization within the body. In individuals with diabetes mellitus, inadequate insulin secretion and/or inadequate insulin action results in high blood glucose concentrations. Treatments for diabetes mellitus focus on maintaining glucose within a target range to avoid its associated symptoms and to minimize the risk of diabetes-related complications over time.

Hypoglycemia can have both acute and long-term consequences. Acute consequences may include seizure, coma, and even death in severe instances. Long-term consequences may include cognitive impairment, poor glucose control with diabetic complications, and impaired hypoglycemia awareness. Acute symptoms include racing heart, anxiety, shaking, irritability, feelings of hunger, weakness, tiredness, dizziness, headache, confusion, and trouble concentrating. Often, hypoglycemia may cause behavioral responses and/or changes in a person's quality of life. This may include reducing the insulin dose, increasing carbohydrate intake, decreasing physical activity, increasing the monitoring of blood glucose, fear, anxiety, and depression.

The primary cause of hypoglycemia in diabetes is diabetes medication. Of particular concern are those medications which raise insulin levels independently of blood glucose, such as sulfonylureas and exogenous insulin. Despite improvements in insulin delivery for patients with type 1 (T1D) and type 2 diabetes (T2D), hypoglycemia incidence remains unacceptably high, with significant negative impact on quality of life, glucose control, and potentially morbidity and mortality when hypoglycemia is severe. Patients with T1D and T2D display dysregulated glucagon secretion (the main defense system against hypoglycemia) that begins early in their disease. Decreased glucagon secretion in response to hypoglycemia may partially explain the high rates of hypoglycemia, particularly in patients on insulin.

Glucagon is a peptide hormone secreted by alpha-cells of the pancreas that promotes the breakdown of glycogen to glucose in the liver, leading to the release of glucose into the bloodstream. Its action therefore tends to raise blood glucose levels, counteracting hypoglycemia. Glucagon is released by the pancreas when the concentration of glucose in blood falls below a certain level. Insulin is in some respects the mirror image of glucagon, being released by the pancreas when blood glucose is high and promoting the uptake of glucose from the blood into tissues. Glucagon and insulin thus form a feedback system that, in healthy individuals, keeps blood glucose levels within a narrow range. See Jones et al. (2012) Endocrinology. 153 (3): 1049-54.

Thus, a method for increasing glucagon secretion in response to exogenous insulin-induced hypoglycemia in patients with type 1 or type 2 diabetes, or a method for preventing or treating hypoglycemia in patients with insulin producing tumors, is desirable.

### SUMMARY OF THE INVENTION

Disclosed herein are methods of preventing or treating hypoglycemia in a mammalian patient, particularly a human, having Type 1 or Type 2 diabetes, by the administration of a therapeutically effective amount of a histamine 1 receptor antagonist, a histamine 3 receptor antagonist, or a combination histamine 1/3 receptor antagonist. In certain embodiments, the patient also is administered a serotonin receptor antagonist, e.g., a 5HT1F antagonist or a 5HT2 antagonist.

In certain embodiments, the methods result in an increase in blood glucose level of at least 5, 10, 15, 20, 25, or 30 mg/dL. In certain embodiments, the patient has a blood glucose level of <70, <65, <60, <59, <58, <57, <56, <55, <54, <53, <52, <51, or <50 mg/dL prior to the administration of the histamine 1 receptor antagonist, the histamine 3 receptor antagonist, or the combination histamine 1/3 receptor antagonist.

In certain embodiments, the patient may experience at least one of a decrease in the frequency of hypoglycemic events, an improvement in the patient's HbA1c, or an increase in time each day that the patient's blood glucose level is within a range between 70-180 mg/dl, inclusive.

In certain embodiments, the patient is taking one or more of the following drugs prior to therapy with a histamine receptor antagonist: insulin, a sulfonyl urea, glyburide, glipizide, glimepiri, repaglinide, nateglinide, chlorpropamide, tolazamide, acetohexamide, or tolbutamide.

In certain embodiments, the methods comprise administration of a therapeutically effective amount a histamine 1 receptor antagonist selected from the group consisting of Mepyramine, Chloropyramine, Antazoline, Tripelennamine, Diphenhydramine, Carbinoxamine, Doxylamine, Orphenadrine, Bromazine, Clemastine, Dimenhydrinate, Pheniramine, Chlorphenamine, Dexchlorpheniramine, Dexbrompheniramine, Brompheniramine, Triprolidine, Dimetindene, Cyclizine, Chlorcyclizine, Hydroxyzine, Meclizine, Promethazine, Alimemazine, Cyproheptadine, Astemizole, Ketotifen Cetirizine, Loratadine, Rupatadine, Mizolastine, Acrivastine, Ebastine, Bilastine, Bepotastine, Terfenadine, Quifenadine, Levocetirizine, Desloratadine, and Fexofenadine.

In certain embodiments, the methods comprise administration of a therapeutically effective amount a histamine 3 receptor antagonist is Pitolisant, Bavisant, Irdabisant, betahistine, thioperamide, AZD5213, ABT239, GSK89254, GSK207040, GSK334429, JNJ-10181457, MK-3134, or MK-0249.

In certain embodiments, the methods comprise administration of a therapeutically effective amount a combination histamine 1/3 receptor antagonist is GSK835726 or GSK1004723.

In certain embodiments, the methods comprise administration of a therapeutically effective amount of the compound AZD5213 having the chemical name 4-[(1S,2S)-2-[(4-cyclobutyl-1-piperazinyl)carbonyl)cyclopropyl benzamide and the following structure:

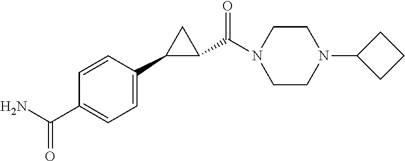 [SMILES: NC(=O)c1ccc([C@H]2C[C@@H]2C(=O)N2CCN(C3CCC3)CC2)cc1]

In certain embodiments, the methods comprise administration of a therapeutically effective amount of a compound disclosed in U.S. Pat. No. 8,063,215, the contents of which are incorporated by reference herein for the purpose of the compounds disclosed therein.

In certain embodiments, the methods comprise administration of a therapeutically effective amount of pitolisant, which has the chemical name 1-{3-[3-(4-chlorophenyl)propoxy]propyl}piperidine and has the following structure:

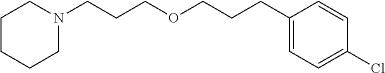 [SMILES: Clc1ccc(CCCOCCCN2CCCCC2)cc1]

In certain embodiments, the methods comprise administration of a therapeutically effective amount of the combination histamine 1/3 receptor antagonist GSK 835726, which has the following structure:

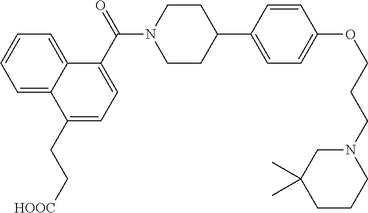 [SMILES: CC1(C)CCCN(CCCOc2ccc(C3CCN(C(=O)c4ccc(CCC(=O)O)c5ccccc45)CC3)cc2)C1]

In certain embodiments, the methods comprise administration of a therapeutically effective amount of the combination histamine 1/3 receptor antagonist GSK 1004723, which has the following structure:

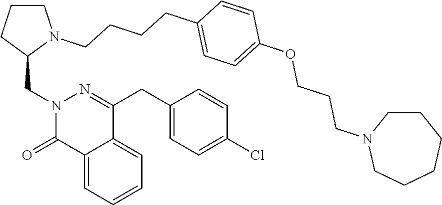 [SMILES: O=c1c2ccccc2c(Cc2ccc(Cl)cc2)nn1C[C@H]1CCCN1CCCCc1ccc(OCCCN2CCCCCC2)cc1]

In certain embodiments, the methods comprise administration of a therapeutically effective amount of a compound disclosed in U.S. Pat. No. 7,989,629, the contents of which are incorporated by reference herein for the purpose of the compounds disclosed therein.

### BRIEF DESCRIPTION OF THE DRAWINGS

FIG. 1 illustrates some of the interrelationships among insulin replacement therapy, Type 1 diabetes, and hypoglycemia.

FIG. 2A illustrates a pancreas with pancreatic islets.

FIG. 2B illustrates structures within a pancreatic islet.

FIG. 3 illustrates histamine suppression of glucagon during hypoglycemia.

FIG. 4 shows how serotonin may cooperate with histamine to suppress glucagon secretion during hypoglycemia.

FIG. 5 shows a timeline for the assays of Examples 1 and 2.

FIG. 6 shows glucagon secretion results for BF2649 hydrochloride and cyproheptadine hydrochloride from Example 1. Mean±SEM from 6 replicates. *p<0.05, **p<0.01, ***p<0.001 at Student's t-test when compared to the vehicle at 1 mM glucose.

FIG. 7 shows results for ATP content per microtissue for BF2649 hydrochloride and cyproheptadine hydrochloride from Example 1. Mean±SEM from 6 replicates. *p<0.05, **p<0.01, ***p<0.001 at Student's t-test when compared to the vehicle at 1 mM glucose.

FIG. 8 shows glucagon secretion results for BF2649 hydrochloride from Example 2. Mean±SEM from 6 replicates. *p<0.05, **p<0.01, ***p<0.001 at Student's t-test when compared to the vehicle at 8 mM glucose.

FIG. 9 shows results for ATP content per microtissue for BF2649 hydrochloride from Example 2. Mean±SEM from 6 replicates. *p<0.05, **p<0.01, ***p<0.001 at Student's t-test when compared to the vehicle at 8 mM glucose.

FIG. 10 shows glucagon secretion results for BF2649 hydrochloride, cetirizine dihydrochloride, and L-arginine from Example 2. Mean±SEM from 6 replicates. *p<0.05, **p<0.01, ***p<0.001 at Student's t-test when compared to the vehicle at 1 mM glucose.

FIG. 11 shows results for ATP content per microtissue for BF2649 hydrochloride, cetirizine dihydrochloride, and L-arginine from Example 2. Mean±SEM from 6 replicates. *p<0.05, **p<0.01, ***p<0.001 at Student's t-test when compared to the vehicle at 1 mM glucose.

### DETAILED DESCRIPTION OF THE INVENTION

A “histamine receptor antagonist,” as used herein, refers to a substance which reduces the action or effect of signaling through a histamine receptor. A histamine receptor antagonist may act via a mechanism that involves binding of the antagonist to the histamine receptor.

A “histamine 1 receptor antagonist,” as used herein, refers to a substance which reduces the action or effect of signaling through the histamine 1 receptor. A histamine 1 receptor antagonist may act via a mechanism that involves binding of the antagonist to the histamine 1 receptor.

A “histamine 3 receptor antagonist,” as used herein, refers to a substance which reduces the action or effect of signaling through the histamine 3 receptor. A histamine 3 receptor antagonist may act via a mechanism that involves binding of the antagonist to the histamine 3 receptor.

A “combination histamine 1/3 receptor antagonist,” as used herein, refers to a single substance (e.g., one particular chemical entity) which reduces the action or effect of signaling through both the histamine 1 receptor and the histamine 3 receptor. A combination histamine 1/3 receptor antagonist may act via a mechanism that involves binding of the antagonist to both the histamine 1 receptor and the histamine 3 receptor.

The histamine receptor antagonists described herein can be specific or non-specific. A “specific” histamine 1 receptor antagonist is a histamine 1 receptor antagonist that does not significantly (statistically) inactivate or reduce the activity of any other type of histamine receptor. For example, a specific histamine 1 receptor antagonist does not reduce the action or effect of the histamine 3 receptor in a statistically significant manner. Similarly, a specific histamine 3 receptor antagonist does not significantly reduce the action or effect of the histamine 1 receptor. In certain embodiments, a specific histamine receptor antagonist also does not reduce the action or effect of receptors other than histamine receptors (e.g., serotonin receptors,)

A “serotonin receptor antagonist,” as used herein, refers to a substance which reduces the action or effect of signaling through a serotonin receptor. A serotonin receptor antagonist may act via a mechanism that involves binding of the antagonist to the serotonin receptor.

A “patient” is a mammal, preferably a human, but can also be companion animals such as dogs or cats, or farm animals such as horses, cattle, pigs, or sheep. In certain embodiments, the patient is a human with renal impairment such as moderate renal impairment. In certain embodiments, the patient is a human child or a human female of child bearing potential.

A “patient in need of therapy for hypoglycemia” refers to a patient that is known to be hypoglycemic or is known to be at risk of becoming hypoglycemic. An example of a patient in need of therapy for hypoglycemia is a Type 1 diabetes patient who is receiving exogenous insulin and who has had at least one episode of hypoglycemia (including severe hypoglycemia) in the previous six months. Other examples include subjects who have had at least one prior episode within the last year, within the last 6 months, and/or within the last month, and in particular, subjects who have had at least 5, 4, 3, or 2 prior episodes of hypoglycemia in those timeframes. Further examples include subjects who have had an episode of severe hypoglycemia that required medical assistance or hospitalization in the last 6 months, and/or subjects who have had a seizure in the last 6 months or is currently having a seizure. Additional examples include subjects who have or have had hypoglycemia unawareness.

A histamine receptor antagonist is said to be administered in a “therapeutically effective amount” if the amount administered results in a desired change in the physiology of the patient, e.g., a reduction in time spent in the hypoglycemic range (which may be, e.g., <70, <65, <60, <59, <58, <57, <56, <55, <54, <53, <52, <51, <50, or <45 mg/dL blood glucose as judged by continuous glucose monitoring, blood glucose monitoring, or laboratory measurements) or a statistically significant reduction in the event rate for all hypoglycemia (e.g., overall, symptomatic, nocturnal). A therapeutically effective amount of a histamine receptor antagonist may be that amount which, when administered to a plurality of patients (e.g., 10, 100, or 1,000) over a suitable time period results in a significant reduction in the percentage of patients with hypoglycemia (including severe hypoglycemia). The efficacy of treatment according to the methods disclosed herein can be monitored by measuring changes in the hypoglycemic event rate before and over time after treatment according to the disclosed methods. Efficacy can also be measured by reduction in severity of hypoglycemia.

The severity of hypoglycemia experienced by an individual patient or a group of patients can be determined by measuring a reduction in the level of hypoglycemia defined as Level 1, glucose<70 mg/dL and glucose≥54 mg/dL; Level 2, glucose<54 mg/dL; and Level 3, a severe event characterized by altered mental and/or physical status requiring assistance. (Agiostratidou Diabetes Care 2017).

A patient “in need of treatment” by the methods disclosed herein does not include a patient being treated with a histamine antagonist where the patient is being treated with the histamine antagonist for a purpose other than to ameliorate hypoglycemia. Thus, a patient in need of treatment by the methods disclosed herein does not include a patient being treated with a histamine receptor antagonist for the purpose of treating allergic rhinitis, allergic conjunctivitis, allergic dermatological conditions (contact dermatitis), rhinorrhea (runny nose), urticaria, angioedema, diarrhea, pruritus (atopic dermatitis, insect bites), anaphylactic or anaphylactoid reactions, nausea or vomiting, sedation, narcolepsy, Alzheimer's disease, attention deficit hyperactivity syndrome, schizophrenia, or pain.

Accordingly, for the purposes of this disclosure, administering a histamine receptor antagonist to a patient “in need of treatment” encompasses only those instances where it is known that it is desirable to ameliorate hypoglycemia in the patient. Thus, such methods do not encompass administering to a patient who happens to be hypoglycemia a therapeutically effective amount of a histamine receptor antagonist for a purpose other than to treat hypoglycemia.

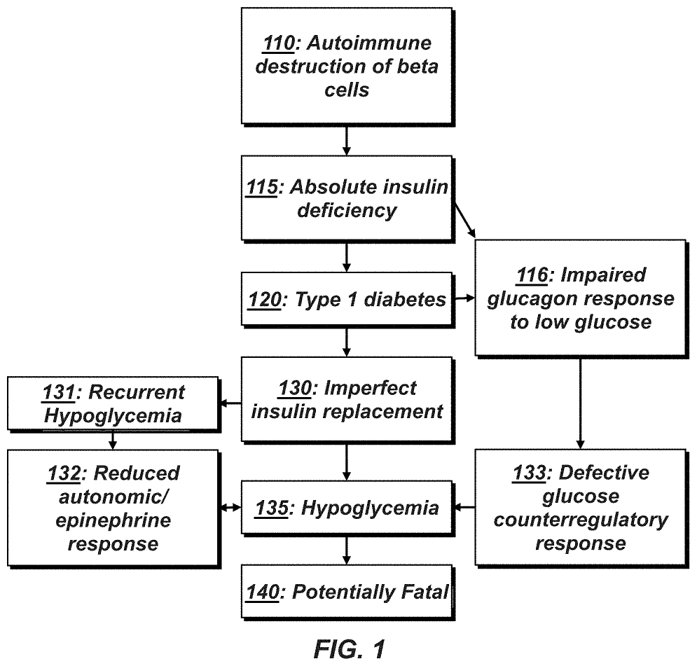

Current insulin replacement therapies are imperfect, with hypoglycemia being a serious problem. Particularly in Type 1 diabetes, the consequences of hypoglycemia can be life threatening, even with advances in diabetes care. As seen in FIG. 1, the autoimmune destruction of beta cells (110) leads to absolute insulin deficiency (115), which in turn may lead to Type 1 diabetes (120). This in turn leads to imperfect insulin replacement (130), which can lead to hypoglycemia (135) and potentially fatal outcomes (140). In some instances, histamines may cause the type 1 diabetic, or individual suffering from absolute insulin deficiency, to have an impaired glucagon response (116) to low glucose. This leads to a defective glucose counterregulatory response (133), which may also lead to hypoglycemia (135). Further, imperfect insulin replacement (130) may lead to recurrent hypoglycemia (131), which results in a reduced autonomic/epinephrine response (132), which makes hypoglycemia (135) more likely to occur in the future.

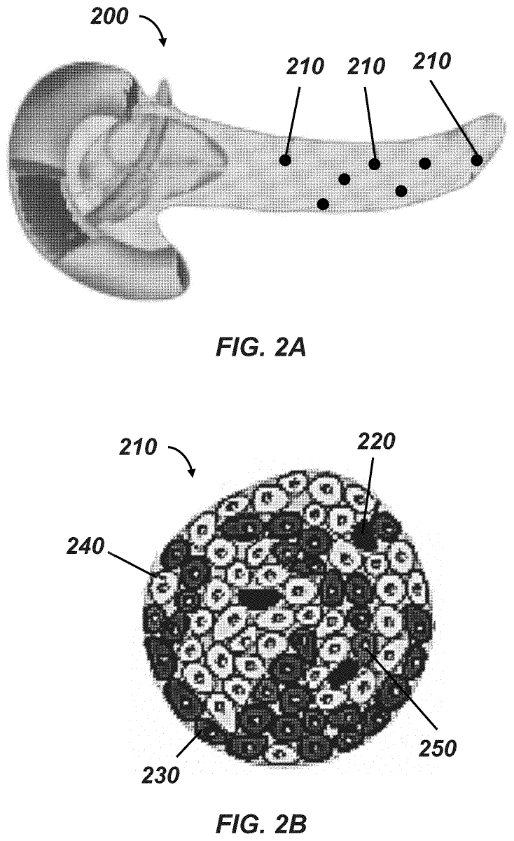

The balance of insulin and glucagon secretion from the pancreas is disrupted in Type 1 diabetes, leading to hyper- and hypoglycemia. Referring to FIGS. 2A and 2B, a pancreas (200) is shown in FIG. 2A. Each pancreas has multiple pancreatic islets (210). Each pancreatic islets contains at least some blood vessels (220), alpha cells (230) (which generate glucagon), beta cells (240) (which generate insulin and amylin) and delta cells (250) (which generate somatostatin). Of the cells in each islet, generally a large percentage are beta cells (240) (typically 50-80% in humans), with fewer alpha cells (230) (typically 15-30% in humans), and relatively few delta cells (typically 4-10% in humans). Islets also generally contain a very small number (typically only 1-2% in humans) of cells that generate pancreatic polypeptide, called pancreatic polypeptide (PP) cells.

In individuals with Type 1 diabetes, many or all of those beta cells (240) are rendered inoperable. This leads to differences in how the islets operate. With normal islets, insulin reduces glucose by reducing hepatic glucose output and increasing glucose uptake into cells. Conversely, glucagon increases blood glucose by increasing hepatic glucose output. In T1D inlets, however, insulin replacement decreases glucose in an unregulated manner. Glucagon secretion is impaired in response to hypoglycemia, even though the alpha cell number remains intact.

As seen in Table 1, below, individuals with diabetes are likely to experience at least one episode of hypoglycemia, and some of those are at risk of experiencing at least one severe episode of hypoglycemia.

TABLE 1
Percentage of patients who experience at least one episode of
hypoglycemia
Incidence rates in all T₁D adult trials (range)
10.4%-12.7% for severe hypoglycemia^{a}
93.0%-99.4% for Novo-Nordisk-defined hypoglycemia^{b}
(basal-bolus regimen)
Incidence rated in all T₂D adult trials (range)
0-4.5% for severe hypoglycemia^{a}
28.5%-80.9% for Novo-Nordisk-defined hypoglycemia^{b} (±OAD or a
basal-bolus regimen)
haps://www.tresibapro.com/clinical-overview/
^{a}Severe hypoglycemia: an event requiring assistance of another person to actively administer carbohydrate, glucagon, or other resuscitative actions.
^{b}Novo Nordisk-defined hypoglycemia: a severe hypoglycemia event or an event where laboratory or self-measured glucose calibrated to plasma was <56 mg/dL or where whole blood glucose was <50 mg/dL (i.e., with or without the presence of hypoglycemic symptoms).

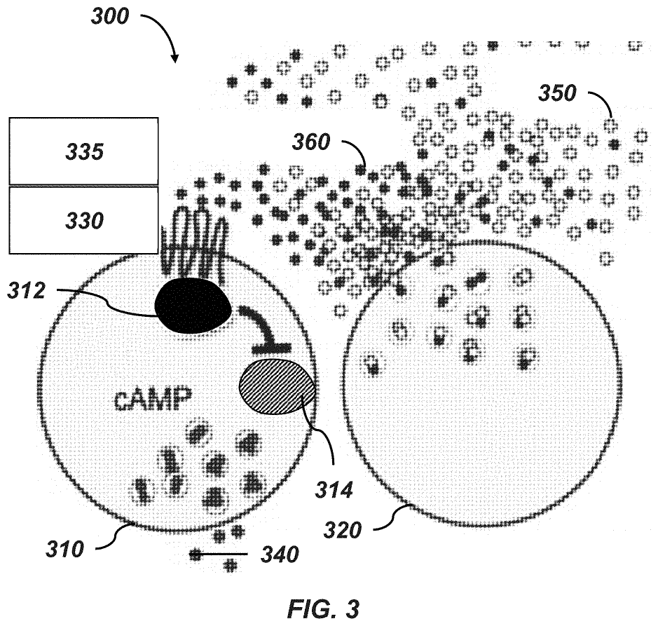

Referring to FIG. 3, In pancreatic islets (300) with alpha cells (310) and beta cells (320), a high level of insulin (350) would normally trigger the release of glucagon (350). T1D and T2D patients are known to not have an increase in glucagon secretion during hypoglycemia. While not wishing to be bound by theory, it is believed histamine (335) either produced locally in the pancreas or derived from the circulation may engage histamine receptors (330) on the alpha-cells (310) to suppress glucagon (340) synthesis and/or secretion during hypoglycemia. Furthermore, serotonin (360) may cooperate with histamine to suppress glucagon secretion during hypoglycemia.

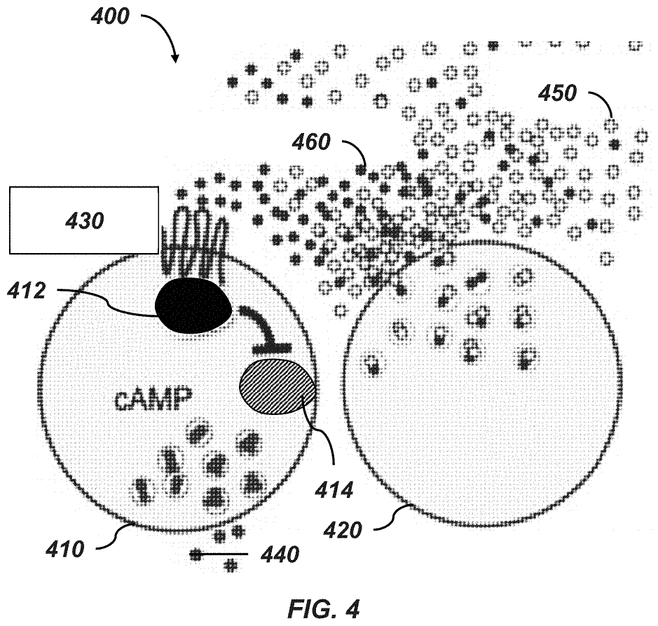

Referring to FIG. 4, in human pancreatic islets (400) with alpha cells (410) and beta cells (420), the beta-cells (420) release serotonin (460) to regulate glucagon (440) secretion and serotonin (460) lowers cyclic adenosine monophosphate (cAMP) in pancreatic alpha-cells (410) via 5HT1F receptors (430).

The present disclosure provides methods of preventing or treating hypoglycemia. The present disclosure also provides methods of restoring normal glucagon secretion in response to insulin induced hypoglycemia in a patient. The present disclosure also provides methods of preventing or treating hypoglycemia in a patient in need of therapy for hypoglycemia without causing hyperglycemia. The methods comprise administering to a patient a therapeutically effective amount of a histamine 1 receptor antagonist, a histamine 3 receptor antagonist, or a combination histamine 1/3 receptor antagonist. In certain embodiments, the methods comprise administering an inverse agonist of the histamine 3 receptor instead of, or in addition to, a histamine 1 receptor antagonist, a histamine 3 receptor antagonist, or a combination histamine 1/3 receptor antagonist. In certain embodiments, the patient is also administered a serotonin receptor antagonist (e.g., a serotonin 5HT1F or 5HT2 antagonist).

Optionally, the method involves treating a subject who has been diagnosed with T1D or T1D. Other embodiments optionally involve preventing hypoglycemia in subjects who have been diagnosed with T1D, T2D, latent autoimmune diabetes in adults (LADA), cystic fibrosis-related diabetes, or diabetes secondary to pancreatectomy. Other embodiments optionally involve preventing hypoglycemia resulting from gastric bypass, reactive or post prandial hypoglycemia, an insulinoma, insulin- or insulin-like growth factor (IGF)-secreting tumors, paraneoplastic conditions associated with hypoglycemia, or other condition associated with hypoglycemia.

Optionally, the patient is a mammal, preferably a human. In certain embodiments, the human has Type 1 diabetes or Type 2 diabetes. In certain embodiments, the patient is receiving exogenous insulin therapy. The insulin therapy may be delivered by methods known in the art (e.g., by multiple daily injections or continuous subcutaneous infusion).

Optionally, the patient has a blood glucose level of <70, <65, <60, <59, <58, <57, <56, <55, <54, <53, <52, <51, <50, or <45 mg/dL prior to therapy with a histamine receptor antagonist.

Optionally, the methods provide an increase in blood glucose level of at least 5, 10, 15, 20, 25, or 30 mg/dL without causing hyperglycemia.

Optionally, the methods do not cause drowsiness and do not significantly affect sleep patterns.

Optionally, the patient may be treated with one or more drugs or medicinal agents prior to therapy with a histamine receptor antagonist. For example, the patient may be treated with insulin, an insulin analog (an altered form of insulin modified to improve some characteristic such as absorption, distribution, metabolism, and excretion, but still able to similarly effect glycemic control), and/or an insulin secretagogue prior to therapy with a histamine receptor antagonist. An insulin secretagogue is a substance that causes the secretion of insulin. Insulin secretagogues can include sulfonyl urea secretagogues and non-sulfonyl urea secretagogues.

Optionally, the patient is taking insulin (in any of its various forms) and/or an insulin analog. In particular embodiments, the patent is taking an insulin or insulin analog selected from human insulin, NPH insulin, insulin lispro, insulin aspart, insulin glulisine, insulin detemir, insulin glargine, insulin degludec, insulin peglispro, insulin known as NN1436 (LA1287), or insulin known as 1218.

Optionally, the patient is being treated with a sulfonyl urea secretagogue by itself or in conjunction with other drugs or medicinal agents. In particular embodiments, the patient is taking a sulfonyl urea secretagogue selected from the group consisting of acetohexamide (e.g., DYMELOR®), carbutamide, chlorpropamide (e.g., DIABINESE®), glibomuride (e.g., GLUTRIL®), gliciazide (e.g., DIAMICRON®), glimepiride (e.g., AMARYL®), glipizide (e.g., GLUCOTROL®), glipquidone (e.g., GLURENORM®), glisoxepide, glyburide (e.g., MICRONASE®), glyclopyramide (e.g., DEAMELIN-S®), tolazamide, (e.g., TOLINASE®), and/or tolbutamide (e.g., ORINASE®).

Optionally, the patient is being treated with a non-sulfonyl urea secretagogue by itself or in conjunction with other drugs or medicinal agents. In particular embodiments, the patient is taking a non-sulfonyl urea secretagogue selected from the group consisting of nateglinide (STARLIX®) and/or repaglinide (PRANDIN®).

Optionally, the patient is being treated with a combination of agents related to reducing blood glucose levels. In particular embodiments, the combination is IDegLira (insulin, degludec, and liraglutide) (e.g., XULTROPHY®).

In certain embodiments, the methods are practiced as long-term therapy, over the course of weeks, months, or years. That is, the patient is administered at least one dose of a histamine 1 receptor antagonist, a histamine 3 receptor antagonist, or a combination histamine 1/3 receptor antagonist for at least several weeks, months, or years. In such embodiments, the methods may provide for a decrease in the frequency of hypoglycemic events or an improvement in the patient's HbA1c or an increase in the amount of time each day that a patient's blood glucose level is between 70-180 mg/dL, inclusive. If the methods are practiced as long term therapy given to a population of patients (i.e., more than 1, 10, 100, 1,000, etc.) the methods may afford a statistically significant improvement in the event rate for all types of hypoglycemia (severe or any) as well as a decrease in the percentage of patients having severe or any hypoglycemia, a decrease in glycemic variability, and/or an improvement in quality of life. In some embodiments, there may be an improvement in impaired hypoglycemia awareness or an improvement in macrovascular or microvascular complications predicated on HbA1c effect size.

Optionally, the present invention relates to methods of treating hypoglycemia where the patient is able to maintain blood glucose levels at or above 70 mg/dl when treated as described herein. In particular embodiments, a patient is able to maintain blood glucose levels at or above 70 mg/dl for a period of 1 week or more, one month or more, six months or more, or one year or more. Optionally, the blood glucose levels in the subject being treated with histamine 1 receptor antagonist, histamine 3 receptor antagonist, and/or combination histamine 1/3 receptor antagonist are increased by at least 5%, 10%, 20%, 30%, 40%, or 50% from the levels prior to treatment. Optionally, the blood glucose levels in the patient are increased by at least 5 mg/dL, 10 mg/dL, 20 mg/dl, 25 mg/dL, 30 mg/dL, 35 mg/dL, 40 mg/dL, 45 mg/dL, or 50 mg/dL.

Optionally, the present invention relates to methods of treating hypoglycemia as described herein where the patient experiences a greater than 5% increase in glucagon

secretion. In specific embodiments, glucagon secretion is increased by 10% or more, 20% or more, or 40% more from the levels prior to treatment. In other embodiments, following treatment, the subject experiences glucagon secretion at a glycemia threshold of at least 10 mg/dL higher than experienced prior to the administration.

It is preferred that the histamine 1 receptor antagonists and the histamine 3 receptor antagonists, as well as the combination histamine 1/3 receptor antagonists, exert their effect on the histamine 1 and 3 receptors in the periphery and do not appreciably function as antagonists of histamine 1 and 3 receptors in the brain. In preferred embodiments therefore, the histamine 1 receptor antagonists and the histamine 3 receptor antagonists, as well as the combination histamine 1/3 receptor antagonists do not cross the blood/brain barrier.

In embodiments where a patient is administered more than one therapeutic agent, e.g., a histamine 1 receptor antagonist and a histamine 3 receptor antagonist, the therapeutic agents may be administered together in a single pharmaceutical composition or separately, each in its own pharmaceutical composition. The frequency of administration and the amount of therapeutic agent in the formulation(s) may vary.

Certain embodiments provide methods where a patient is administered a histamine 1 receptor antagonist or a histamine 3 receptor antagonist in combination with another active pharmaceutical agent where the other active pharmaceutical agent is administered for a purpose unrelated to controlling hypoglycemia but is known to have the undesirable side effect of lowering blood glucose levels. For example, an embodiment is directed to a method for preventing or treating hypoglycemia in a patient taking an agent selected from insulin (in any of its various forms), a sulfonyl urea, glyburide (MICRONASE®), glipizide (GLUCOTROL®), glimepiride (AMARYL®), repaglinide (PRANDIN®), nateglinide (STARLIX®), chlorpropamide (DIABINESE®), tolazamide (TOLINASE®), acetohexamide (DYMELOR®), or tolbutamide (ORINASE®) by administering a histamine 1 receptor antagonist or a histamine 3 receptor antagonist that ameliorates the reduction in blood sugar level caused by the other active pharmaceutical agent, thus alleviating at least some of the undesirable effect on blood sugar levels of the other active pharmaceutical ingredient.

In certain embodiments, the methods disclosed herein comprise the step of identifying a patient in need of therapy for hypoglycemia. Thus, the methods include a method of identifying and treating a patient for hypoglycemia comprising:

(a) identifying a patient in need of therapy for hypoglycemia; and
(b) administering to the patient a therapeutically effective amount of a histamine 1 receptor antagonist or a histamine 3 receptor antagonist or a combination histamine 1/3 receptor antagonist.

In methods such as that disclosed immediately above, “identifying a patient in need of therapy for hypoglycemia” refers to knowingly selecting for treatment such a patient. That is, such methods do not encompass administering to the patient a therapeutically effective amount of a histamine 1 receptor antagonist or a histamine 3 receptor antagonist or a combination histamine 1/3 receptor antagonist where the patient is selected for such administration not because the patient is hypoglycemic, or is at risk of becoming hypoglycemic, but instead because the patient has some other medical condition. Thus, such methods do not encompass administering to a patient who happens to be hypoglycemic (but is not known to be hypoglycemic) a therapeutically effective amount of a histamine 1 receptor antagonist or a histamine 3 receptor antagonist or a combination histamine 1/3 receptor antagonist for a purpose other than to treat hypoglycemia. Such methods encompass only the administration of a histamine 1 receptor antagonist or a histamine 3 receptor antagonist or a combination histamine 1/3 receptor antagonist for the purpose of preventing or treating hypoglycemia.

In certain embodiments, the patient has been selected for administration of a histamine 1 receptor antagonist or a histamine 3 receptor antagonist or a combination histamine 1/3 receptor antagonist because the patient has been identified as having a blood glucose level of <70, <65, <60, <59, <58, <57, <56, <55, <54, <53, <52, <51, <50, or <45 mg/dL.

Therapeutic agents disclosed herein may be formulated into pharmaceutical compositions. The therapeutic agents may be present in the pharmaceutical compositions in the form of salts of pharmaceutically acceptable acids or in the form of bases. The therapeutic agents may be present in amorphous form or in crystalline forms, including hydrates and solvates.

Pharmaceutically acceptable salts of the therapeutic agents disclosed herein include salts derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate salts.

Salts derived from appropriate bases include alkali metal (e.g., sodium and potassium), alkaline earth metal (e.g., magnesium), ammonium and N⁺(C₁₋₄alkyl)₄ salts.

The therapeutic agents are also meant to include (unless specified otherwise) all stereochemical forms of the therapeutic agents (i.e., the R and S configurations for each asymmetric center). Therefore, single enantiomers, racemic mixtures, and diastereomers of the therapeutic agents are contemplated. Also contemplated are steric isomers and positional isomers of the therapeutic agents. The therapeutic agents are also meant to include compounds which differ only in the presence of one or more isotopically enriched atoms. For example, therapeutic agents in which one or more hydrogens are replaced by deuterium or tritium, or the replacement of one or more carbons by ¹³C- or ¹⁴C-enriched carbon are contemplated.

In some embodiments, the therapeutic agents disclosed herein are administered in a pharmaceutical composition that includes a pharmaceutically acceptable carrier, adjuvant, excipient, or vehicle. The term “pharmaceutically acceptable carrier, adjuvant, excipient, or vehicle” refers to a non-toxic carrier, adjuvant, excipient, or vehicle that does not destroy or significantly diminish the pharmacological activity of the therapeutic agent with which it is formulated. Pharmaceutically acceptable carriers, adjuvants, excipients, or vehicles that may be used in the compositions encompass any of the standard pharmaceutically accepted liquid carriers, such as a phosphate-buffered saline solution, water, as well as emulsions such as an oil/water emulsion or a triglyceride emulsion. Solid carriers may include excipients such as starch, milk, sugar, certain types of clay, stearic acid, talc, gums, glycols, or other known excipients. Carriers may also include flavor and color additives as well as other ingredients.

The pharmaceutical compositions are preferably administered orally, preferably once daily as solid compositions, taken at any time of the day with or without food. However, the pharmaceutical compositions may be administered parenterally, by inhalation spray, topically, rectally, nasally, buccally, vaginally, or via an implanted reservoir. Sterile injectable forms of the pharmaceutical compositions may be aqueous or oleaginous suspensions. These suspensions may be formulated according to techniques known in the art using suitable dispersing, wetting, or suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

The pharmaceutical compositions may be orally administered in any orally acceptable dosage form, including, but not limited to, solid forms such as capsules and tablets or liquids. In the case of tablets for oral use, carriers commonly used include microcrystalline cellulose, lactose and corn starch. Lubricating agents, such as magnesium stearate, are also typically added. When aqueous suspensions are required for oral use, the active ingredient may be combined with emulsifying and suspending agents. If desired, certain sweetening, flavoring or coloring agents may also be added.

The pharmaceutical compositions may also be administered by nasal aerosol or inhalation. Such pharmaceutical compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Should topical administration be desired, it can be accomplished using any method commonly known to those skilled in the art and includes but is not limited to incorporation of the pharmaceutical composition into creams, ointments, or transdermal patches.

In addition to the common dosage forms set out above, the compounds may also be administered by controlled release means and/or delivery devices. Various controlled release means and/or delivery devices are known in the art.

Additional therapeutic agents, which are normally administered to treat hypoglycemia may also be present in the pharmaceutical compositions disclosed herein or may be administered concurrently with the pharmaceutical compositions disclosed herein. In some embodiments, the patient is a diabetes patient undergoing continuous or intermittent glucose monitoring who is also undergoing a diabetes educational program such as a program relating to the diabetes patient's activity, diet, and/or insulin self-management. In some embodiments, the patient is undergoing treatment with an artificial pancreas such as the Medtronic MINIMED® 670G Insulin Pump System. A patient may also be a diabetes patient who has undergone islet cell transplantation. In some embodiments, the patient is also administered an antagonist or an inverse agonist of a somatostatin type 2 receptor. In some embodiments, the patient is also acutely administered glucagon for the treatment of severe hypoglycemia.

In certain embodiments, provided are methods where a patient is administered a histamine 1 receptor antagonist or a histamine 3 receptor antagonist or a combination histamine 1/3 receptor antagonist and no other active pharmaceutical ingredient. In some embodiments, the patient is administered no other substance known to be effective for the treatment of hypoglycemia other than a histamine 1 receptor antagonist or a histamine 3 receptor antagonist or a combination histamine 1/3 receptor antagonist.

The amount of histamine 1 receptor antagonist or histamine 3 receptor antagonist or a combination histamine 1/3 receptor antagonist that may be combined with carrier materials to produce a pharmaceutical composition in a single dosage form will vary depending upon the patient treated and the particular mode of administration. It should be understood that a specific dosage and treatment regimen for any particular patient may depend upon a variety of factors, including: the activity of the specific antagonist employed; the age, body weight, general health, sex, and diet of the patient; the time of administration of the antagonist; the rate of excretion of the antagonist; the severity of the particular condition being treated; as well as the judgment of the treating physician. Despite their variety, accounting for these factors in order to select an appropriate dosage or treatment regimen would require no more than routine experimentation and is therefore well within the ordinary skill in the art.

The amount of antagonist to be administered in the present methods depends on many factors, as discussed above. However, in humans, for example, the amount generally ranges from about 0.1 mg/day to about 2 g/day; preferably from about 0.5 mg/day to about 500 mg/day; or from about 20 mg/day to about 250 mg/day; or from about 40 mg/day to about 100 mg/day. Other preferred dosages include about 2 mg/day, about 5 mg/day, about 10 mg/day, about 15 mg/day, about 20 mg/day, about 25 mg/day, about 30 mg/day, about 40 mg/day, about 50 mg/day, about 60 mg/day, about 70 mg/day, about 80 mg/day, about 90 mg/day, about 100 mg/day, about 125 mg/day, about 150 mg/day, about 175 mg/day, about 200 mg/day, about 250 mg/day, about 300 mg/day, about 350 mg/day, about 400 mg/day, about 500 mg/day, about 600 mg/day, about 700 mg/day, about 800 mg/day, and about 900 mg/day. Routine experimentation will determine the appropriate value for each patient by monitoring the effect of the antagonist(s) on patient blood glucose level, which can be frequently and easily monitored. The antagonist can be administered once or multiple times per day. The frequency of administration may vary from a single dose per day to multiple doses (1, 2, 3, 4, or more) per day. The daily dosage regimen will preferably be from 0.01 to 200 mg/kg, 0.05 to 175 mg/kg, 0.1 to 150 mg/kg, 0.5 to 100 mg/kg, or 1 to 75 mg/kg, of total body weight.

In certain embodiments, the antagonist or combinations thereof are repeatedly administered to the patient and the patient's blood glucose level is measured until it is increased to a desired level. For example, in certain embodiments, the patient's blood glucose level is increased by at least 5, 10, 15, 20, 25, or 30 mg/dL compared to the patient's blood glucose level prior to the first administration of the antagonist.

While it is desirable to raise blood glucose level in a patient with hypoglycemia, it is undesirable to raise blood glucose level to the extent that hyperglycemia ensues. The present methods raise a patient's blood glucose level and thus treat hypoglycemia without also causing hyperglycemia. By acting in accord with the body's normal feedback system for controlling glucagon secretion from the pancreas, the methods relieve the inhibition of glucagon release due to excess histamine or serotonin but do not lead to the release of glucagon in an amount that would lead to hyperglycemia.

A preferred patient is a human with an elevated plasma histamine level. Such a level includes plasma histamine levels of >200 pg/mL, >250 pg/mL, >300 pg/mL, >350 pg/ml, >400 pg/mL, >450 pg/mL, or >500 pg/mL. Such a level includes plasma histamine levels of 200 pg/mL to 300 pg/mL; 300 pg/mL to 400 pg/mL; 400 pg/mL to 500 pg/mL; or 500 pg/mL to 600 pg/mL. In some embodiments, the patient is a patient with Type 1 or Type 2 diabetes with an elevated plasma histamine level.

There is the potential that suppression of glucagon secretion during hypoglycemia may involve peripheral serotonin (5HT), which will also be a potential target by blocking serotonin receptors on alpha cells to increase glucagon to prevent hypoglycemia. Specifically, 5HT1F and/or 5HT2 receptor blockers may be effective for hypoglycemia prevention through restoring normal glucagon secretion.

Various histamine 1 receptor antagonists are considered suitable for the methods disclosed herein, including but not limited to Mepyramine, Chloropyramine, Antazoline, Tripelennamine, Diphenhydramine, Carbinoxamine, Doxylamine, Orphenadrine, Bromazine, Clemastine, Dimenhydrinate, Pheniramine, Chlorphenamine, Dexchlorpheniramine, Dexbrompheniramine, Brompheniramine, Triprolidine, Dimetindene, Cyclizine, Chlorcyclizine, Hydroxyzine, Meclizine, Promethazine, Alimemazine, Cyproheptadine, Astemizole, Ketotifen Cetirizine, Loratadine, Rupatadine, Mizolastine. Acrivastine, Ebastine, Bilastine, Bepotastine, Terfenadine, Quifenadine, Levocetirizine, Desloratadine, and Fexofenadine.

Histamine 3 receptor antagonists that may be used include, but are not limited to, Pitolisant, Bavisant, Irdabisant, betahistine, thioperamide, AZD5213, ABT239, GSK189254, GSK207040, GSK334429, JNJ-10181457, MK-3134 and MK-0249. Combination histamine 1/3 receptor antagonists that may be used include, but are not limited to, GSK835726 and GSK1004723.

5HT receptor antagonists that may be used include, but are not limited to, GSK127935, Chlorpromazine, Cyproheptadine, Metergoline, Methysergide, Mianserin, Mirtazapine, Oxetorone, Pizotifen, Ritanserin, and Spiperone.

In some embodiments of the methods disclosed herein, the histamine 3 receptor antagonist is AZD5213, which has the chemical name 4-[(S,2S)-2-[(4-cyclobutyl-1-piperazinyl)carbonyl)cyclopropyl benzamide and has the following structure:

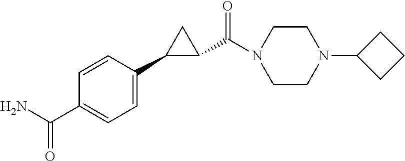 [SMILES: NC(=O)c1ccc([C@H]2C[C@@H]2C(=O)N2CCN(C3CCC3)CC2)cc1]

In some embodiments of the methods disclosed herein, the histamine 3 receptor antagonist is a compound disclosed in U.S. Pat. No. 8,063,215, the contents of which are incorporated by reference herein for the purpose of the compounds disclosed therein.

In some embodiments of the methods disclosed herein, the histamine 3 receptor antagonist is pitolisant, which has the chemical name 1-{3-[3-(4-chlorophenyl)propoxy]propyl}piperidine and has the following structure:

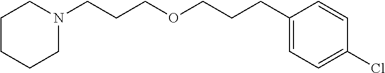 [SMILES: Clc1ccc(CCCOCCCN2CCCCC2)cc1]

In some embodiments the methods disclosed herein employ the combination histamine 1/3 receptor antagonist GSK 835726, which has the following structure:

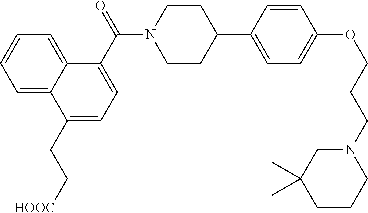 [SMILES: CC1(C)CCCN(CCCOc2ccc(C3CCN(C(=O)c4ccc(CCC(=O)O)c5ccccc45)CC3)cc2)C1]

In some embodiments the methods disclosed herein employ the combination histamine 1/3 receptor antagonist GSK 1004723, which has the following structure:

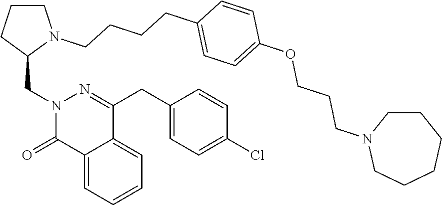 [SMILES: O=c1c2ccccc2c(Cc2ccc(Cl)cc2)nn1C[C@H]1CCCN1CCCCc1ccc(OCCCN2CCCCCC2)cc1]

In some embodiments of the methods disclosed herein, the histamine 1 receptor, histamine 1/3 receptor antagonist, or combination histamine 1/3 receptor antagonist is a compound disclosed in U.S. Pat. No. 7,989,629, the contents of which are incorporated by reference herein for the purpose of the compounds disclosed therein.

In various embodiments, the treatment may be provided as a kit, and specifically, a kit comprising a container that includes (1) a pharmaceutical composition comprising a pharmaceutically acceptable carrier and a histamine 1 receptor antagonist, a histamine 3 receptor antagonist, and/or a combination histamine 1/3 receptor antagonist; and (2) printed instructions for preventing or treating hypoglycemia in a patient as described herein.

In the present specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. The contents of all references, pending patent applications and published patents, cited throughout this application are hereby expressly incorporated by reference as if set forth herein in their entirety, except where terminology is not consistent with the definitions herein. Although specific terms are employed, they are used as in the art unless otherwise indicated.

### EXAMPLES

### Example 1—Histamine Blockade Results in Increased Glucagon Secretion in a Diabetes Model System Under Hypoglycemic Conditions

This and the following example make use of 3D INSIGHT® Islet Microtissues (InSphero AG, Schlieren, Switzerland), an in vitro model system for diabetes research produced by optimized dissociation and controlled scaffold-free reaggregation of primary human pancreatic islet cells. This allows precise control over the newly forming islet microtissue size and eliminates contaminating exocrine material while ensuring homogeneous and native-like distribution of endocrine cells within each tissue. The resulting islet tissues display long-term (>28 days) and robust function, enabling high throughput and longitudinal study of pancreatic islet function, regulation, and preservation.

Histamine antagonists were tested under hypoglycemic/insulinemic conditions. Three different concentrations of the histamine 3 receptor antagonist BF2649 hydrochloride (pitolisant) and the histamine 1 receptor antagonist cyproheptadine hydrochloride were tested with regards to their effect on acute glucagon secretion.

Details of the assay were as follows:

1. The diabetes model system 3D INSIGHT® Islet Microtissues was obtained from a donor.
2. Glucagon secretion was observed for 2 hours.
3. Six replicates for each sample were tested.
4. L-arginine was used as a positive control.
5. DMSO was used a solvent for BF2649 hydrochloride and cyproheptadine hydrochloride; ddH₂O was used as a solvent for L-arginine. All experimental groups were treated with similar concentrations of DMSO to homogenize potentially adverse effects of the solvent.
6. Glucagon secretion was quantified with a glucagon ELISA (Mercodia, Uppsala, Sweden).
7. Total ATP content was also measured during the assay.

TABLE 2
Donor information

| UNOS ID # | AFBM114 |
|---|---|
| Age | 28 |
| Sex | Male |
| Race | Caucasian |
| BMI | 34.7 |
| Positive serologies | EBV IgG, CMV IgG |
| HbA_{1c} | 4.2% |
| Cause of death | Blunt injuries |
| Mechanism of death | Head trauma |
| Islets classified normal or T₂ | Normal |

TABLE 3
Islet microtissue information

| | |
|---|---|
| Microtissue type | Human pancreatic islet |
| Cell composition | Primary human pancreatic endocrine cells |

TABLE 4
Quality test results for islet microtissue

| Parameter | Specification | Result |
|---|---|---|
| Average diameter | 136-181 μm | 173 μm ± 12 μm |
| CV % diameter | <10 | 7.0 |
| Average IEQ | 0.75-1.75 | 1.555 ± 0.313 |
| ATP-content | >2 pmol ATP/IEQ | 2.24 ± 0.30 pmol ATP/IEQ |
| GSIS: basic glucose | Detectable | 0.064 ± 0.013 fmol/min*IEQ⁻¹ |
| (2.8 mM) | | |
| GSIS: high glucose | >0.2 fmol/min* | 1.608 ± 0.293 fmol/min*IEQ⁻¹ |
| (16.8 mM) | IEQ⁻¹ | |
| Stimulation index | >2-fold | 25 |

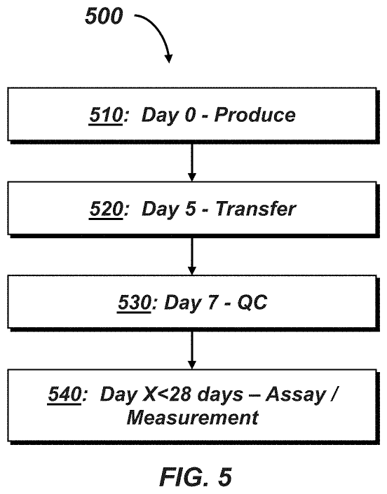

Referring to FIG. 5, a timeline (500) of the assay is shown. Day 0 (510) involved producing the 3D INSIGHT® Human Islet Microtissues. On Day 5 (520), the samples were moved to a receiving plate. On Day 7 (530), the islet size, viability, and function were verified by quality control. On the final day of the test (540), the glucagon secretion assay was performed with ELISA, and ATP content was measured.

Before the assay, conditioned media was removed, islet microtissue washed twice with Kreb's Ringer HEPES buffer (KRHB) containing 5.5 mM glucose and were equilibrated in KRHB containing 5-5 mM glucose for 1.5 hours. Equilibration buffer was removed, islet microtissues were washed twice in KRHB with 5-5 mM glucose. Glucagon secretion was performed in KRHB with either 8 mM or 1 mM glucose for 2 hours in the presence of the test compounds. Conditioned KRHB were collected for analysis of glucagon content with an ELISA (Mercodia, Uppsala, Sweden). The tissues were lysed to analyze total ATP content using a CELLTITER-GLO® Luminescent Cell Viability Assay (Promega, Madison, Wis.).

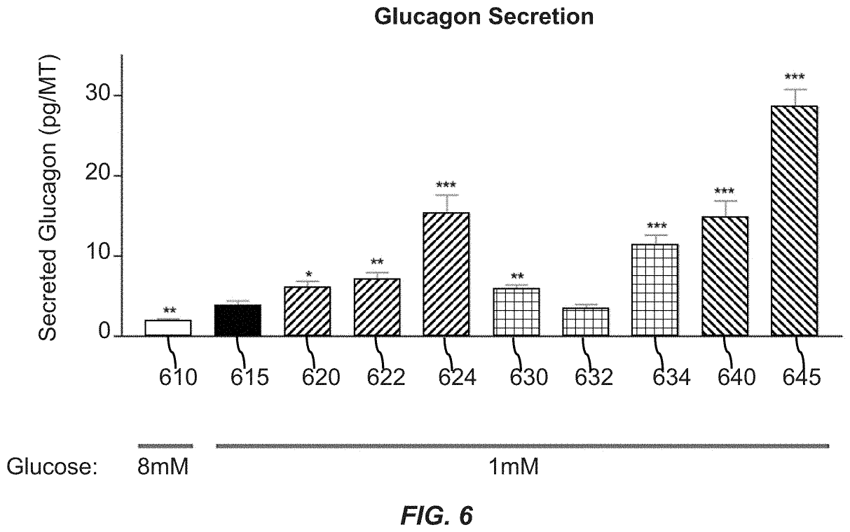

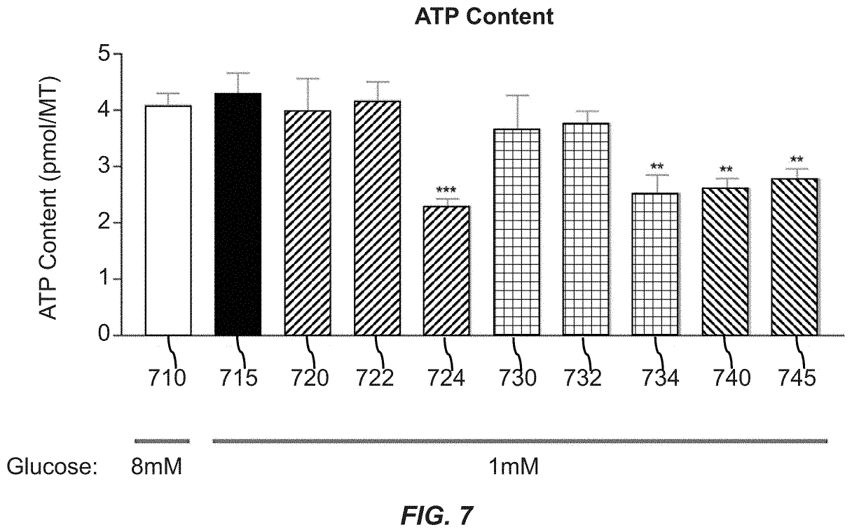

Results for glucagon secretion are shown in FIG. 6, and total ATP content are shown in FIG. 7. FIGS. 6 and 7 including the vehicle (solvent) controls at 8 mM glucose (610, 710) and 1 mM glucose (615, 715), as well as for BF 2649 hydrochloride at 1 μM (620, 720), 10 μM (622, 722), and 100 μM (624, 724); Cyproheptadine hydrochloride at 1 μM (630, 730), 10 μM (632, 732), and 100 μM (634, 734); and L-Arginine at 5 mM (640, 740) and 10 mM (645, 745). FIG. 6 provides mean±SEM from 6 replicates, where * indicates a p<0.05 (see 620), ** indicates a p<0.01 (see 610, 622, 630), and *** indicates a p<0.001 (see 624, 634, 640, 645) at Student's t-test when compared to the vehicle at 1 mM glucose. FIG. 7 provides mean±SEM from 6 replicates, where ** indicates a p<0.01 (see 734, 740, 745), and *** indicates a p<0.001 (see 724 at Student's t-test when compared to the vehicle at 1 mM glucose.

As can be seen in FIGS. 6 and 7, the histamine 3 receptor antagonists BF2649 hydrochloride and the histamine 1 receptor antagonist cyproheptadine hydrochloride, as well as the positive control L-arginine, increased glucagon secretion, and the increase was higher at 1 mM glucose than at 8 mM glucose. For BF2649, the increase was dose dependent. The increase in glucagon secretion appears to be accompanied by a decrease in ATP levels for 100 μM BF2649 and cyproheptadine, and for 5 and 10 mM L-arginine.

Statistical methods were as follows:

All test result values are reported as mean±standard error of the mean (SEM). Statistical significance was determined with a Student's t-test, rejecting the null hypothesis at p=0.05. Results are represented with *p<0.05, **p<0.01, and ***p<0.001. Outliers were detected with Grubb's outlier test (alpha=0.05).

### Example 2—Additional Data Showing that Histamine Blockade Results in Increased Glucagon Secretion in a Diabetes Model System Under Hypoglycemic Conditions

This example, like Example 1, used the 3D INSIGHT® Islet Microtissues model system for diabetes research to further test the ability of the histamine 3 antagonist BF2649 hydrochloride to increase glucagon release from human pancreatic islet cells. Also tested was cetirizine dihydrochloride, a histamine 1 antagonist. This example used the same islet cell donor as Example 1 as well as an additional donor.

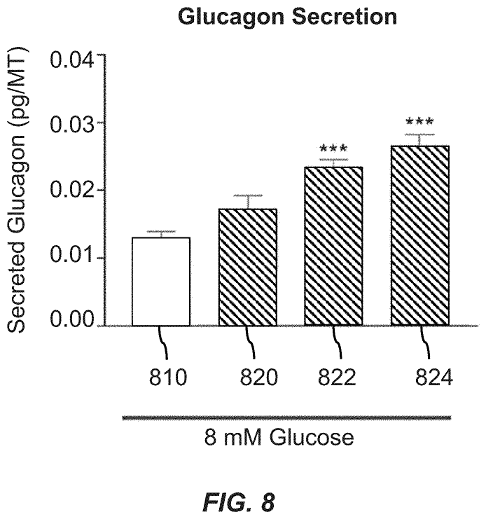

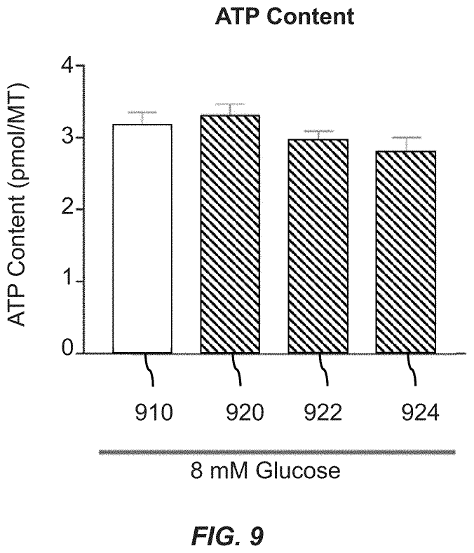

Details of the assay were the same for this example as for Example 1 except that cetirizine dihydrochloride, a selective, non-brain penetrant histamine 1 receptor antagonist, was tested instead of cyproheptadine hydrochloride. The donor with UNOS ID #AFBM114 was also used in this study shown in FIGS. 8 and 9. A second donor (UNOS ID #AFCS437) was used as well.

TABLE 5
Second donor information

| | |
|---|---|
| UNOS ID # | AFCS₄₃₇ |
| Age | 46 |
| Sex | Male |
| Race | Hispanic |
| BMI | 33.2 |
| Positive serologies | EBV IgG, CMV IgG |
| HbA_{1c} | 5.4% |
| Cause of death | Anoxia |
| Mechanism of death | Stroke |
| Islets classified normal or T₂ | Normal |

TABLE 6
Second donor islet microtissue information

| | |
|---|---|
| Microtissue type | Human pancreatic islet |
| Cell composition | Primary human pancreatic endocrine cells |

TABLE 7
Second donor quality test results for islet microtissue

| Parameter | Specification | Result |
|---|---|---|
| Average diameter | 136-181 μm | 165 μm ± 5.0 μm |
| CV % diameter | <10 | 3.0 |
| Average IEQ | 0.75-1.75 | 1.337 ± 0.120 |
| ATP-content | >2 pmol ATP/IEQ | 3.03 ± 0.35 pmol ATP/IEQ |
| GSIS: basic glucose | Detectable | 0.015 ± 0.003 fmol/min*IEQ⁻¹ |
| (2.8 mM) | | |
| GSIS: high glucose | >0.2 fmol/min* | 0.988 ± 0.273 fmol/min*IEQ⁻¹ |
| (16.8 mM) | IEQ⁻¹ | |
| Stimulation index | >2-fold | 64.7 |

The timeline for this example was the same as that for Example 1 (see FIG. 5).

Before the assay, conditioned media was removed, islet microtissue washed twice with Kreb's Ringer HEPES buffer (KRHB) containing 5-5 mM glucose and were equilibrated in KRHB containing 5-5 mM glucose for 1.5 hours. Equilibration buffer was removed, islet microtissues were washed twice in KRHB with 5-5 mM glucose. Glucagon secretion was performed in KRHB with either 8 mM or 1 mM glucose for 2 hours in the presence of the test compounds. Conditioned KRHB were collected for analysis of glucagon content with an ELISA (Mercodia, Uppsala, Sweden). The tissues were lysed to analyze total ATP content using a CELLTITER-GLO® Luminescent Cell Viability Assay (Promega, Madison, Wis.).

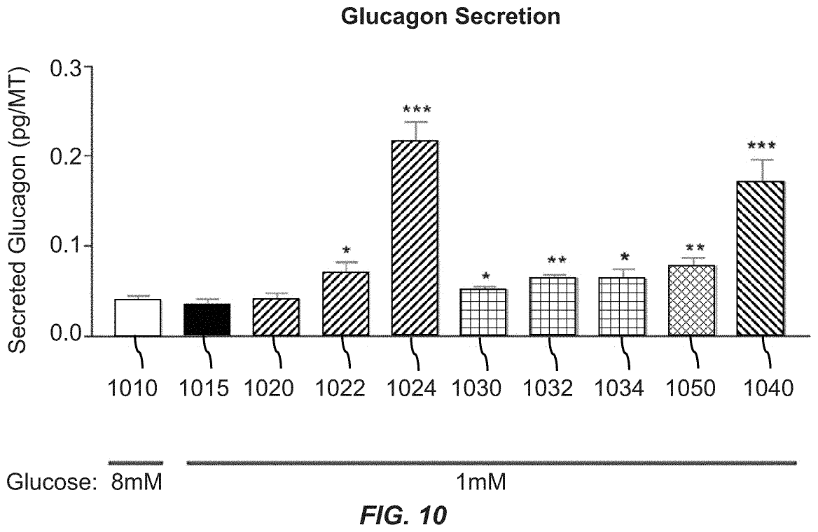

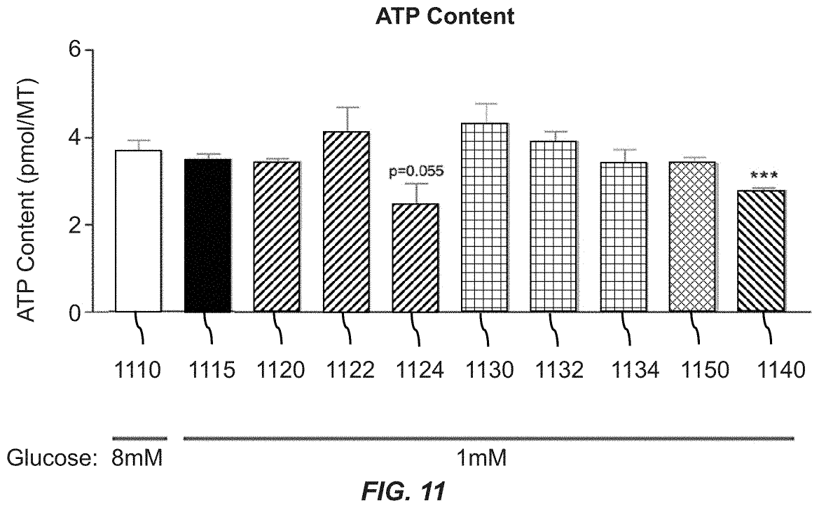

Results for glucagon secretion are shown in FIG. 8 for BF2649 and in FIG. 10 for BF2649 and cetirizine. Results for total ATP content are shown in FIG. 9 for BF2649 and in FIG. 11 for BF2649 and cetirizine.

FIGS. 8 and 9 include the vehicle (solvent) controls at 8 mM glucose (810, 910), as well as for BF 2649 hydrochloride at 1 μM (820, 920), 10 μM (822, 922), and 100 μM (824, 924). FIGS. 10 and 11 include the vehicle (solvent) controls at 8 mM (1010, 1110) and 1 mM glucose (1015, 1115), as well as for BF 2649 hydrochloride at 1 μM (1020, 1120), 10 μM (1022, 1122), and 100 μM (1024, 1124); Cyproheptadine hydrochloride at 1 μM (1030, 1130), 10 μM (1032, 1132), and 100 μM (1034, 1134); L-Arginine at 5 mM (1040, 1140); and a combination treatment (10 μM BF2649+10 μM cetirizine) (1050, 1150).

For BF2649 and donor AFBM114, glucagon secretion at 8 mM glucose (0.012 nM) was very close to the level detected with the same donor as was used in Example 1. BF2649 increased glucagon secretion at 10 μM and 100 μM at 8 mM glucose. Although the fold induction was similar at 10 μM in both glucose concentrations (1.8 fold), at 100 μM BF2649 increased glucagon secretion significantly more when used with 1 mM glucose (7.5 fold in Example 1) compared to 8 mM glucose (2 fold).

For donor AFCS437, glucagon secretion at 1 mM glucose was not higher than at 8 mM glucose but all the tested compounds increased glucagon secretion. BF2649 displayed a significant dose dependency in its potency to increase glucagon secretion. As was observed for the previously tested donor, 100 μM BF2649 increased glucagon secretion similarly to 5 mM arginine. The increase in glucagon secretion appeared to be accompanied by a decrease in ATP levels for 100 μM of BF2649 (p=0.55) and for 5 mM L-arginine. Cetirizine only slightly increased glucagon secretion at all tested concentrations, and its dose dependency was much less pronounced. And lastly, the glucagon response to the combination treatment (10 μM BF2649+10 μM cetirizine) was similar in magnitude to 10 μM B2649 alone and only slightly higher than 10 μM cetirizine alone.

Statistical methods were as in Example 1.

The data obtained in the above examples indicate that histamine 3 receptor antagonists or inverse agonists may reduce the incidence of hypoglycemia without increasing the risk of hyperglycemia. This is because such antagonists or agonists are able to significantly increase glucagon secretion in the hypoglycemic state (i mM glucose) but have only minimal effect on glucagon secretion under normoglycemic conditions (8 mM glucose). (Compare FIGS. 6 and 8).

Various modifications and variations of the invention in addition to those shown and described herein will be apparent to those skilled in the art without departing from the scope and spirit of the invention and fall within the scope of the claims. Although the invention has been described in connection with specific preferred embodiments, it should be understood that the invention as claimed should not be unduly limited to such specific embodiments.

In addition, the references listed herein are also part of the application and are incorporated by reference in their entirety as if fully set forth herein.

1. A method for restoring normal glucagon secretion comprising administering to a patient known to have Type 1 diabetes or Type 2 diabetes a therapeutically effective amount of at least one antagonist selected from the group consisting of a histamine 1 receptor antagonist, a histamine 3 receptor antagonist, and a combination histamine 1/3 receptor antagonist, in response to insulin induced hypoglycemia in the patient.

2. A method according to claim 1, wherein the administration of the therapeutically effective amount of at least one antagonist prevents or treats hypoglycemia in patients in need of therapy for hypoglycemia, or in a patient known to have Type 1 diabetes, Type 2 diabetes, or an insulin producing tumor.

3. The method according to claim 1, wherein the patient is administered both a histamine 1 receptor antagonist and a histamine 3 receptor antagonist.

4. The method according to claim 1, wherein the patient is administered a combination histamine 1/3 receptor antagonist.

5. The method according to claim 1, wherein the patient is administered a therapeutic amount of a serotonin receptor antagonist.

6. The method according to claim 5, wherein the serotonin receptor antagonist is a 5HT1F antagonist or a 5HT2 antagonist.

7. The method according to claim 1, wherein the patient's blood glucose level increases by at least 5, 10, 15, 20, 25, or 30 mg/dL.

8. The method according to claim 1, wherein the patient has a blood glucose level of <70, <65, <60, <59, <58, <57, <56, <55, <54, <53, <52, <51, or <50 mg/dL prior to the administration of the histamine 1 receptor antagonist, the histamine 3 receptor antagonist, or the combination histamine 1/3 receptor antagonist.

9. The method according to claim 1, wherein the histamine 1 receptor antagonist is Mepyramine, Chloropyramine, Antazoline, Tripelennamine, Diphenhydramine, Carbinoxamine, Doxylamine, Orphenadrine, Bromazine, Clemastine, Dimenhydrinate, Pheniramine, Chlorphenamine, Dexchlorpheniramine, Dexbrompheniramine, Brompheniramine, Triprolidine, Dimetindene, Cyclizine, Chlorcyclizine, Hydroxyzine, Meclizine, Promethazine, Alimemazine, Cyproheptadine, Astemizole, Ketotifen Cetirizine, Loratadine, Rupatadine, Mizolastine, Acrivastine, Ebastine, Bilastine, Bepotastine, Terfenadine, Quifenadine, Levocetirizine, Desloratadine, or Fexofenadine.

10. The method according to claim 1, wherein the histamine 3 receptor antagonist is Pitolisant, Bavisant, Irdabisant, betahistine, thioperamide, AZD5213, ABT239, GSK189254, GSK207040, GSK334429, JNJ-10181457, MK-3134, or MK-0249.

11. The method according to claim 1, wherein the combination histamine 1/3 receptor antagonist is GSK835726 or GSK1004723.

12. The method according to claim 1, wherein the patient is taking one or more of the following drugs prior to therapy with a histamine receptor antagonist: insulin, a sulfonyl urea, glyburide, glipizide, glimepiride, repaglinide, nateglinide, chlorpropamide, tolazamide, acetohexamide, or tolbutamide.

13. The method according to claim 1, wherein the patient is administered a pharmaceutical composition comprising a histamine 1 receptor antagonist, a histamine 3 receptor antagonist, or a combination histamine 1/3 receptor antagonist at least once per day for at least one week.

14. The method according to claim 1, wherein the patient experiences a decrease in the frequency of hypoglycemic events.
